SEQUENCE LISTING (253 sequences)

SEQ ID NO: 1 - protein, 6 aa, Gallus gallus
FGLSGL

SEQ ID NO: 2 - protein, 7 aa, Gallus gallus
AAVGSSL

SEQ ID NO: 3 - protein, 7 aa, Gallus gallus
TAVGSST

SEQ ID NO: 4 - protein, 6 aa, Gallus gallus
LSLSSQ

SEQ ID NO: 5 - protein, 7 aa, Gallus gallus
FGISGLG

SEQ ID NO: 6 - protein, 6 aa, Gallus gallus
PILSSF

SEQ ID NO: 7 - protein, 7 aa, Gallus gallus
SLLSQGA

SEQ ID NO: 8 - protein, 6 aa, Gallus gallus
FNLRGA

SEQ ID NO: 9 - protein, 8 aa, Gallus gallus
SAAVGSSL

SEQ ID NO: 10 - protein, 6 aa, Gallus gallus
SRVVIQ

SEQ ID NO: 11 - protein, 6 aa, Gallus gallus
SRVVLE

SEQ ID NO: 12 - protein, 8 aa, Gallus gallus
GFGISGLG

SEQ ID NO: 13 - protein, 6 aa, Gallus gallus
SRFSGR

SEQ ID NO: 14 - protein, 6 aa, Gallus gallus
RVVIQP

SEQ ID NO: 15 - protein, 8 aa, Gallus gallus
SAAVGSIL

SEQ ID NO: 16 - protein, 7 aa, Gallus gallus
IVVIGHV

SEQ ID NO: 17 - protein, 9 aa, Gallus gallus
TAVGSSTSA

SEQ ID NO: 18 - protein, 7 aa, Gallus gallus
RVVIQPS

SEQ ID NO: 19 - protein, 7 aa, Gallus gallus
SRVVIQP

SEQ ID NO: 20 - protein, 7 aa, Gallus gallus
PQGEQLQ

SEQ ID NO: 21 - protein, 9 aa, Gallus gallus
RAGAGPPSP

SEQ ID NO: 22 - protein, 7 aa, Gallus gallus
DSRVVIQ

SEQ ID NO: 23 - protein, 8 aa, Gallus gallus
PGPILSSF

SEQ ID NO: 24 - protein, 9 aa, Gallus gallus
PISSGGFGI

SEQ ID NO: 25 - protein, 8 aa, Gallus gallus
ISGLGSRF

SEQ ID NO: 26 - protein, 10 aa, Gallus gallus
SGGFGISGLG

SEQ ID NO: 27 - protein, 7 aa, Gallus gallus
SRFSGRR

SEQ ID NO: 28 - protein, 9 aa, Gallus gallus
PGLALPVLP

SEQ ID NO: 29 - protein, 8 aa, Gallus gallus
SRVVIQPS

SEQ ID NO: 30 - protein, 8 aa, Gallus gallus
PILSSFPQ

SEQ ID NO: 31 - protein, 8 aa, Gallus gallus
LGSGIFWI

SEQ ID NO: 32 - protein, 9 aa, Gallus gallus
GISGLGSRF

SEQ ID NO: 33 - protein, 9 aa, Gallus gallus
VVVTLPGPI

SEQ ID NO: 34 - protein, 8 aa, Gallus gallus
CRLSLSQP

SEQ ID NO: 35 - protein, 11 aa, Gallus gallus
SAAVGSSLSAG

SEQ ID NO: 36 - protein, 11 aa, Gallus gallus
AVGSSTSAAVG

SEQ ID NO: 37 - protein, 9 aa, Gallus gallus
VVTLPGPIL

SEQ ID NO: 38 - protein, 9 aa, Gallus gallus
PGPILSSFP

SEQ ID NO: 39 - protein, 10 aa, Gallus gallus
SAAVGSILSE

SEQ ID NO: 40 - protein, 10 aa, Gallus gallus
VPISSGGFGI

SEQ ID NO: 41 - protein, 8 aa, Gallus gallus
QLAQRNTI

SEQ ID NO: 42 - protein, 9 aa, Gallus gallus
LSGLRSNLS

SEQ ID NO: 43 - protein, 10 aa, Gallus gallus
PSPVVVTLPG

SEQ ID NO: 44 - protein, 10 aa, Gallus gallus
PPPVVVTLPG

SEQ ID NO: 45 - protein, 10 aa, Gallus gallus
ISGLGSRFSG

SEQ ID NO: 46 - protein, 9 aa, Gallus gallus
LALRQSVEA

SEQ ID NO: 47 - protein, 10 aa, Gallus gallus
PLLGPPLLGI

SEQ ID NO: 48 - protein, 10 aa, Gallus gallus
VVTLPGPILS

SEQ ID NO: 49 - protein, 8 aa, Gallus gallus
VTLEDLRR

SEQ ID NO: 50 - protein, 9 aa, Gallus gallus
PILSSFPQN

SEQ ID NO: 51 - protein, 9 aa, Gallus gallus
PILSSFPQD

SEQ ID NO: 52 - protein, 11 aa, Gallus gallus
GTVGVSAGVTR

SEQ ID NO: 53 - protein, 9 aa, Gallus gallus
FGLSRVTLN

SEQ ID NO: 54 - protein, 12 aa, Gallus gallus
TAVGSSTSAAVG

SEQ ID NO: 55 - protein, 10 aa, Gallus gallus
VVVTLPGPIL

SEQ ID NO: 56 - protein, 10 aa, Gallus gallus
PPVVVTFPGP

SEQ ID NO: 57 - protein, 10 aa, Gallus gallus
PPPVVVTFPG

SEQ ID NO: 58 - protein, 10 aa, Gallus gallus
VVIQPSPVVV

SEQ ID NO: 59 - protein, 10 aa, Gallus gallus
FGISGLGSRF

SEQ ID NO: 60 - protein, 10 aa, Gallus gallus
PGPILSSFPQ

SEQ ID NO: 61 - protein, 10 aa, Gallus gallus
FGWSGLGRGL

SEQ ID NO: 62 - protein, 9 aa, Gallus gallus
LEAQLSELR

SEQ ID NO: 63 - protein, 9 aa, Gallus gallus
IVGAWYTVR

SEQ ID NO: 64 - protein, 11 aa, Gallus gallus
LLSPPGLPGLL

SEQ ID NO: 65 - protein, 11 aa, Gallus gallus
VVVTLPGPILS

SEQ ID NO: 66 - protein, 11 aa, Gallus gallus
GFGISGLGSRF

SEQ ID NO: 67 - protein, 11 aa, Gallus gallus
PVVVTLPGPIL

SEQ ID NO: 68 - protein, 11 aa, Gallus gallus
VSQGEITVPSI

SEQ ID NO: 69 - protein, 11 aa, Gallus gallus
SILSEEGVPIS

SEQ ID NO: 70 - protein, 11 aa, Gallus gallus
PVVVTFPGPIL

SEQ ID NO: 71 - protein, 13 aa, Gallus gallus
PISSGGFGISGLG

SEQ ID NO: 72 - protein, 13 aa, Gallus gallus
VGSSTSAAVGSIL

SEQ ID NO: 73 - protein, 11 aa, Gallus gallus
GFLSSAVSLNR

SEQ ID NO: 74 - protein, 12 aa, Gallus gallus
GGFGISGLGSRF

SEQ ID NO: 75 - protein, 11 aa, Gallus gallus
PGPILSSFPQN

SEQ ID NO: 76 - protein, 11 aa, Gallus gallus
PGPILSSFPQD

SEQ ID NO: 77 - protein, 12 aa, Gallus gallus
GGFGWSGLGRGL

SEQ ID NO: 78 - protein, 12 aa, Gallus gallus
PSPVVVTLPGPI

SEQ ID NO: 79 - protein, 12 aa, Gallus gallus
VVVTLPGPILSS

SEQ ID NO: 80 - protein, 12 aa, Gallus gallus
PVVVTLPGPILS

SEQ ID NO: 81 - protein, 12 aa, Gallus gallus
SPVVVTLPGPIL

SEQ ID NO: 82 - protein, 11 aa, Gallus gallus
RVVIQPSPVVV

SEQ ID NO: 83 - protein, 11 aa, Gallus gallus
QALTEVYAKAN

SEQ ID NO: 84 - protein, 10 aa, Gallus gallus
IETLTQFLGW

SEQ ID NO: 85 - protein, 13 aa, Gallus gallus
GFGSRSLVGLGGT

SEQ ID NO: 86 - protein, 14 aa, Gallus gallus
AVGSSTSAAVGSIL

SEQ ID NO: 87 - protein, 12 aa, Gallus gallus
PVVVTFPGPILS

SEQ ID NO: 88 - protein, 14 aa, Gallus gallus
VGSSTSAAVGSILS

SEQ ID NO: 89 - protein, 13 aa, Gallus gallus
GFGISGLGSRFSG

SEQ ID NO: 90 - protein, 13 aa, Gallus gallus
SGGFGISGLGSRF

SEQ ID NO: 91 - protein, 14 aa, Gallus gallus
VPISSGGFGISGLG

SEQ ID NO: 92 - protein, 12 aa, Gallus gallus
VVIQPSPVVVTL

SEQ ID NO: 93 - protein, 13 aa, Gallus gallus
SILSEEGVPISSG

SEQ ID NO: 94 - protein, 13 aa, Gallus gallus
VMGCKAAGASRII

SEQ ID NO: 95 - protein, 13 aa, Gallus gallus
SPVVVTLPGPILS

SEQ ID NO: 96 - protein, 12 aa, Gallus gallus
SRVVIQPSPVVV

SEQ ID NO: 97 - protein, 13 aa, Gallus gallus
PSPVVVTLPGPIL

SEQ ID NO: 98 - protein, 12 aa, Gallus gallus
RVVIQPSPVVVT

SEQ ID NO: 99 - protein, 13 aa, Gallus gallus
PPPVVVTLPGPIL

SEQ ID NO: 100 - protein, 15 aa, Gallus gallus
GVPISSGGFGISGLG

SEQ ID NO: 101 - protein, 13 aa, Gallus gallus
VIQPSPVVVTLPG

SEQ ID NO: 102 - protein, 15 aa, Gallus gallus
AVGSSTSAAVGSILS

SEQ ID NO: 103 - protein, 15 aa, Gallus gallus
TAVGSSTSAAVGSIL

SEQ ID NO: 104 - protein, 14 aa, Gallus gallus
SGGFGISGLGSRFS

SEQ ID NO: 105 - protein, 13 aa, Gallus gallus
VVVTLPGPILSSF

SEQ ID NO: 106 - protein, 13 aa, Gallus gallus
PPPVVVTFPGPIL

SEQ ID NO: 107 - protein, 14 aa, Gallus gallus
GLGGFGWSGLGRGL

SEQ ID NO: 108 - protein, 13 aa, Gallus gallus
VTLPGPILSSFPQ

SEQ ID NO: 109 - protein, 14 aa, Gallus gallus
AVGSILSEEGVPIS

SEQ ID NO: 110 - protein, 14 aa, Gallus gallus
SLGLGGFGWSGLGR

SEQ ID NO: 111 - protein, 14 aa, Gallus gallus
PSPVVVTLPGPILS

SEQ ID NO: 112 - protein, 13 aa, Gallus gallus
SRVVIQPSPVVVT

SEQ ID NO: 113 - protein, 15 aa, Gallus gallus
SGGFGISGLGSRFSG

SEQ ID NO: 114 - protein, 14 aa, Gallus gallus
PPPVVVTLPGPILS

SEQ ID NO: 115 - protein, 14 aa, Gallus gallus
VAIQPPPVVVTLPG

SEQ ID NO: 116 - protein, 15 aa, Gallus gallus
PISSGGFGISGLGSR

SEQ ID NO: 117 - protein, 13 aa, Gallus gallus
PIVDVAEALLHVK

SEQ ID NO: 118 - protein, 14 aa, Gallus gallus
VVIQPSPVVVTLPG

SEQ ID NO: 119 - protein, 13 aa, Gallus gallus
RVVIQPSPVVVTL

SEQ ID NO: 120 - protein, 16 aa, Gallus gallus
TAVGSSTSAAVGSILS

SEQ ID NO: 121 - protein, 14 aa, Gallus gallus
PPPVVVTFPGPILS

SEQ ID NO: 122 - protein, 15 aa, Gallus gallus
SLGLGGFGWSGLGRG

SEQ ID NO: 123 - protein, 14 aa, Gallus gallus
VVVTLPGPILSSFP

SEQ ID NO: 124 - protein, 14 aa, Gallus gallus
PVVVTLPGPILSSF

SEQ ID NO: 125 - protein, 15 aa, Gallus gallus
AAVGSILSEEGVPIS

SEQ ID NO: 126 - protein, 15 aa, Gallus gallus
SAAVGSILSEEGVPI

SEQ ID NO: 127 - protein, 16 aa, Gallus gallus
EGVPISSGGFGISGLG

SEQ ID NO: 128 - protein, 14 aa, Gallus gallus
VVTLPGPILSSFPQ

SEQ ID NO: 129 - protein, 15 aa, Gallus gallus
PSPVVVTLPGPILSS

SEQ ID NO: 130 - protein, 16 aa, Gallus gallus
GKDGPKGVRGDAGPPG

SEQ ID NO: 131 - protein, 15 aa, Gallus gallus
SVAIQPPPVVVTLPG

SEQ ID NO: 132 - protein, 14 aa, Gallus gallus
SRVVIQPSPVVVTL

SEQ ID NO: 133 - protein, 14 aa, Gallus gallus
LHTEAAATVPDRSE

SEQ ID NO: 134 - protein, 15 aa, Gallus gallus
VVIQPSPVVVTLPGP

SEQ ID NO: 135 - protein, 14 aa, Gallus gallus
RVVIQPSPVVVTLP

SEQ ID NO: 136 - protein, 16 aa, Gallus gallus
SAAVGSILSEEGVPIS

SEQ ID NO: 137 - protein, 15 aa, Gallus gallus
PVVVTLPGPILSSFP

SEQ ID NO: 138 - protein, 15 aa, Gallus gallus
IQPSPVVVTLPGPIL

SEQ ID NO: 139 - protein, 17 aa, Gallus gallus
SVPIGAGGSLGLGGFGW

SEQ ID NO: 140 - protein, 16 aa, Gallus gallus
SLGLGGFGWSGLGRGL

SEQ ID NO: 141 - protein, 17 aa, Gallus gallus
FGGGFGGGDGILPAGEK

SEQ ID NO: 142 - protein, 17 aa, Gallus gallus
TAVGSSTSAAVGSILSE

SEQ ID NO: 143 - protein, 16 aa, Gallus gallus
PISSGGFGISGLGSRF

SEQ ID NO: 144 - protein, 19 aa, Gallus gallus
SAAVGSSLSAGSVPIGAGG

SEQ ID NO: 145 - protein, 17 aa, Gallus gallus
GVPISSGGFGISGLGSR

SEQ ID NO: 146 - protein, 15 aa, Gallus gallus
VVVTLPGPILSSFPQ

SEQ ID NO: 147 - protein, 15 aa, Gallus gallus
RVVIQPSPVVVTLPG

SEQ ID NO: 148 - protein, 15 aa, Gallus gallus
SRVVIQPSPVVVTLP

SEQ ID NO: 149 - protein, 16 aa, Gallus gallus
PSPVVVTLPGPILSSF

SEQ ID NO: 150 - protein, 16 aa, Gallus gallus
AIQPPPVVVTLPGPIL

SEQ ID NO: 151 - protein, 16 aa, Gallus gallus
IQPSPVVVTLPGPILS

SEQ ID NO: 152 - protein, 16 aa, Gallus gallus
VIQPSPVVVTLPGPIL

SEQ ID NO: 153 - protein, 17 aa, Gallus gallus
VPISSGGFGISGLGSRF

SEQ ID NO: 154 - protein, 16 aa, Gallus gallus
SRVVIQPSPVVVTLPG

SEQ ID NO: 155 - protein, 17 aa, Gallus gallus
SILSEEGVPISSGGFGI

SEQ ID NO: 156 - protein, 16 aa, Gallus gallus
PVVVTLPGPILSSFPQ

SEQ ID NO: 157 - protein, 16 aa, Gallus gallus
RVVIQPSPVVVTLPGP

SEQ ID NO: 158 - protein, 18 aa, Gallus gallus
SAAVGSILSEEGVPISSG

SEQ ID NO: 159 - protein, 16 aa, Gallus gallus
VVVTLPGPILSSFPQN

SEQ ID NO: 160 - protein, 18 aa, Gallus gallus
GVPISSGGFGISGLGSRF

SEQ ID NO: 161 - protein, 17 aa, Gallus gallus
PSPVVVTLPGPILSSFP

SEQ ID NO: 162 - protein, 17 aa, Gallus gallus
VAIQPPPVVVTLPGPIL

SEQ ID NO: 163 - protein, 17 aa, Gallus gallus
VIQPSPVVVTLPGPILS

SEQ ID NO: 164 - protein, 17 aa, Gallus gallus
VVIQPSPVVVTLPGPIL

SEQ ID NO: 165 - protein, 17 aa, Gallus gallus
SPVVVTLPGPILSSFPQ

SEQ ID NO: 166 - protein, 15 aa, Gallus gallus
VILSNTKTQYSIKIH

SEQ ID NO: 167 - protein, 17 aa, Gallus gallus
SRVVIQPSPVVVTLPGP

SEQ ID NO: 168 - protein, 17 aa, Gallus gallus
DSRVVIQPSPVVVTLPG

SEQ ID NO: 169 - protein, 17 aa, Gallus gallus
PVVVTLPGPILSSFPQN

SEQ ID NO: 170 - protein, 17 aa, Gallus gallus
RVVIQPSPVVVTLPGPI

SEQ ID NO: 171 - protein, 19 aa, Gallus gallus
GVPISSGGFGISGLGSRFS

SEQ ID NO: 172 - protein, 18 aa, Gallus gallus
SVAIQPPPVVVTLPGPIL

SEQ ID NO: 173 - protein, 18 aa, Gallus gallus
VAIQPPPVVVTLPGPILS

SEQ ID NO: 174 - protein, 18 aa, Gallus gallus
VVIQPSPVVVTLPGPILS

SEQ ID NO: 175 - protein, 18 aa, Gallus gallus
PSPVVVTLPGPILSSFPQ

SEQ ID NO: 176 - protein, 20 aa, Gallus gallus
GVPISSGGFGISGLGSRFSG

SEQ ID NO: 177 - protein, 18 aa, Gallus gallus
AVPLVRPVTMVPSIPGIP

SEQ ID NO: 178 - protein, 18 aa, Gallus gallus
PPPVVVTLPGPILSSFPQ

SEQ ID NO: 179 - protein, 18 aa, Gallus gallus
SPVVVTLPGPILSSFPQN

SEQ ID NO: 180 - protein, 18 aa, Gallus gallus
LPGPGLQLLHGTTTLAFK

SEQ ID NO: 181 - protein, 18 aa, Gallus gallus
PPPVVVTFPGPILSSFPQ

SEQ ID NO: 182 - protein, 19 aa, Gallus gallus
SVAIQPPPVVVTLPGPILS

SEQ ID NO: 183 - protein, 19 aa, Gallus gallus
SSVAIQPPPVVVTLPGPIL

SEQ ID NO: 184 - protein, 18 aa, Gallus gallus
RVVIQPSPVVVTLPGPIL

SEQ ID NO: 185 - protein, 19 aa, Gallus gallus
PSPVVVTLPGPILSSFPQN

SEQ ID NO: 186 - protein, 21 aa, Gallus gallus
SILSEEGVPISSGGFGISGLG

SEQ ID NO: 187 - protein, 19 aa, Gallus gallus
SRVVIQPSPVVVTLPGPIL

SEQ ID NO: 188 - protein, 19 aa, Gallus gallus
RVVIQPSPVVVTLPGPILS

SEQ ID NO: 189 - protein, 22 aa, Gallus gallus
SVPIGAGGSLGLGGFGWSGLGR

SEQ ID NO: 190 - protein, 22 aa, Gallus gallus
TAVGSSTSAAVGSILSEEGVPI

SEQ ID NO: 191 - protein, 22 aa, Gallus gallus
SAAVGSILSEEGVPISSGGFGI

SEQ ID NO: 192 - protein, 21 aa, Gallus gallus
SEEGVPISSGGFGISGLGSRF

SEQ ID NO: 193 - protein, 20 aa, Gallus gallus
SRVVIQPSPVVVTLPGPILS

SEQ ID NO: 194 - protein, 23 aa, Gallus gallus
SVPIGAGGSLGLGGFGWSGLGRG

SEQ ID NO: 195 - protein, 20 aa, Gallus gallus
DSRVVIQPSPVVVTLPGPIL

SEQ ID NO: 196 - protein, 23 aa, Gallus gallus
TAVGSSTSAAVGSILSEEGVPIS

SEQ ID NO: 197 - protein, 22 aa, Gallus gallus
LRGDVGPVGRTGEQGIAGPPGF

SEQ ID NO: 198 - protein, 21 aa, Gallus gallus
SRVVIQPSPVVVTLPGPILSS

SEQ ID NO: 199 - protein, 21 aa, Gallus gallus
VVIQPSPVVVTLPGPILSSFP

SEQ ID NO: 200 - protein, 22 aa, Gallus gallus
GVPISSGGFGISGLGSRFSGRR

SEQ ID NO: 201 - protein, 24 aa, Gallus gallus
SVPIGAGGSLGLGGFGWSGLGRGL

SEQ ID NO: 202 - protein, 21 aa, Gallus gallus
DSRVVIQPSPVVVTLPGPILS

SEQ ID NO: 203 - protein, 21 aa, Gallus gallus
VIQPSPVVVTLPGPILSSFPQ

SEQ ID NO: 204 - protein, 24 aa, Gallus gallus
AVGSILSEEGVPISSGGFGISGLG

SEQ ID NO: 205 - protein, 21 aa, Gallus gallus
RVVIQPSPVVVTLPGPILSSF

SEQ ID NO: 206 - protein, 23 aa, Gallus gallus
SILSEEGVPISSGGFGISGLGSR

SEQ ID NO: 207 - protein, 21 aa, Gallus gallus
IPSSVPESSFKIKGNSSSFHI

SEQ ID NO: 208 - protein, 22 aa, Gallus gallus
VAIQPPPVVVTLPGPILSSFPQ

SEQ ID NO: 209 - protein, 25 aa, Gallus gallus
TAVGSSTSAAVGSILSEEGVPISSG

SEQ ID NO: 210 - protein, 22 aa, Gallus gallus
VVIQPSPVVVTLPGPILSSFPQ

SEQ ID NO: 211 - protein, 23 aa, Gallus gallus
PSPVVVTLPGPILSSFPQNTAVG

SEQ ID NO: 212 - protein, 20 aa, Gallus gallus
KENFEVLCKDGTRKPVTDAE

SEQ ID NO: 213 - protein, 21 aa, Gallus gallus
SIQACKLAQSHGWGVMVSHRS

SEQ ID NO: 214 - protein, 22 aa, Gallus gallus
SRVVIQPSPVVVTLPGPILSSF

SEQ ID NO: 215 - protein, 22 aa, Gallus gallus
RVVIQPSPVVVTLPGPILSSFP

SEQ ID NO: 216 - protein, 23 aa, Gallus gallus
EQPPLCVASVSVKEPASETPAVP

SEQ ID NO: 217 - protein, 23 aa, Gallus gallus
SVAIQPPPVVVTLPGPILSSFPQ

SEQ ID NO: 218 - protein, 26 aa, Gallus gallus
SAAVGSILSEEGVPISSGGFGISGLG

SEQ ID NO: 219 - protein, 24 aa, Gallus gallus
SILSEEGVPISSGGFGISGLGSRF

SEQ ID NO: 220 - protein, 24 aa, Gallus gallus
PSPVVVTLPGPILSSFPQNTAVGS

SEQ ID NO: 221 - protein, 23 aa, Gallus gallus
VVIQPSPVVVTLPGPILSSFPQN

SEQ ID NO: 222 - protein, 23 aa, Gallus gallus
SRVVIQPSPVVVTLPGPILSSFP

SEQ ID NO: 223 - protein, 24 aa, Gallus gallus
SSVAIQPPPVVVTLPGPILSSFPQ

SEQ ID NO: 224 - protein, 23 aa, Gallus gallus
RVVIQPSPVVVTLPGPILSSFPQ

SEQ ID NO: 225 - protein, 25 aa, Gallus gallus
SILSEEGVPISSGGFGISGLGSRFS

SEQ ID NO: 226 - protein, 27 aa, Gallus gallus
TSAAVGSILSEEGVPISSGGFGISGLG

SEQ ID NO: 227 - protein, 26 aa, Gallus gallus
SILSEEGVPISSGGFGISGLGSRFSG

SEQ ID NO: 228 - protein, 24 aa, Gallus gallus
SRVVIQPSPVVVTLPGPILSSFPQ

SEQ ID NO: 229 - protein, 28 aa, Gallus gallus
TAVGSSTSAAVGSILSEEGVPISSGGFG

SEQ ID NO: 230 - protein, 28 aa, Gallus gallus
STSAAVGSILSEEGVPISSGGFGISGLG

SEQ ID NO: 231 - protein, 24 aa, Gallus gallus
RVVIQPSPVVVTLPGPILSSFPQN

SEQ ID NO: 232 - protein, 25 aa, Gallus gallus
SRVVIQPSPVVVTLPGPILSSFPQN

SEQ ID NO: 233 - protein, 25 aa, Gallus gallus
DSRVVIQPSPVVVTLPGPILSSFPQ

SEQ ID NO: 234 - protein, 29 aa, Gallus gallus
TAVGSSTSAAVGSILSEEGVPISSGGFGI

SEQ ID NO: 235 - protein, 29 aa, Gallus gallus
SAAVGSILSEEGVPISSGGFGISGLGSRF

SEQ ID NO: 236 - protein, 24 aa, Gallus gallus
VEQLVNLQHLDVAYNLLLEHAQLA

SEQ ID NO: 237 - protein, 28 aa, Gallus gallus
SVAIQPPPVVVTLPGPILSSFPQSTAVG

SEQ ID NO: 238 - protein, 28 aa, Gallus gallus
SILSEEGVPISSGGFGISGLGSRFSGRR

SEQ ID NO: 239 - protein, 30 aa, Gallus gallus
TSAAVGSILSEEGVPISSGGFGISGLGSRF

SEQ ID NO: 240 - protein, 29 aa, Gallus gallus
SSVAIQPPPVVVTLPGPILSSFPQSTAVG

SEQ ID NO: 241 - protein, 31 aa, Gallus gallus
STSAAVGSILSEEGVPISSGGFGISGLGSRF

SEQ ID NO: 242 - protein, 31 aa, Gallus gallus
PSPVVVTLPGPILSSFPQNTAVGSSTSAAVG

SEQ ID NO: 243 - protein, 33 aa, Gallus gallus
TAVGSSTSAAVGSILSEEGVPISSGGFGISGLG

SEQ ID NO: 244 - protein, 31 aa, Gallus gallus
AVGSILSEEGVPISSGGFGISGLGSRFSGRR

SEQ ID NO: 245 - protein, 27 aa, Gallus gallus
NKVLVHCAMGRSRSATLVLAYLMIYKN

SEQ ID NO: 246 - protein, 32 aa, Gallus gallus
SSVAIQPPPVVVTLPGPILSSFPQSTAVGSTA

SEQ ID NO: 247 - protein, 32 aa, Gallus gallus
AAVGSILSEEGVPISSGGFGISGLGSRFSGRR

SEQ ID NO: 248 - protein, 35 aa, Gallus gallus
TAVGSSTSAAVGSILSEEGVPISSGGFGISGLGSR

SEQ ID NO: 249 - protein, 33 aa, Gallus gallus
SAAVGSILSEEGVPISSGGFGISGLGSRFSGRR

SEQ ID NO: 250 - protein, 34 aa, Gallus gallus
PSPVVVTLPGPILSSFPQNTAVGSSTSAAVGSIL

SEQ ID NO: 251 - protein, 35 aa, Gallus gallus
PSPVVVTLPGPILSSFPQNTAVGSSTSAAVGSILS

SEQ ID NO: 252 - protein, 36 aa, Gallus gallus
TAVGSSTSAAVGSILSEEGVPISSGGFGISGLGSRF

SEQ ID NO: 253 - protein, 29 aa, Gallus gallus
RPRVYLHCTNPRHPKYREGDVTLFALNLS

(19) United States
(12) Patent Application Publication
(10) Pub. No.: US 2025/0084010 A1
(43) Pub. Date: Mar. 13, 2025

(54) METHOD OF USING/APPLYING A KERATIN HYDROLYSIS PEPTIDE SOLUTION UPON GRAPE PLANTS TO INCREASE PRODUCTION YIELD

(71) Applicant: CH Biotech R&D Co., Ltd., Nantou City (TW)

(72) Inventors: Kai XIA, Nantou (TW); Ching-Chi TSAI, Taichung City (TW); Meng-Ying LI, Nantou (TW); Yu-Lun LIU, Taichung (TW); Nai-Hua YE, Changhua (TW)

(73) Assignee: CH Biotech R&D Co., Ltd., Nantou City (TW)

(21) Appl. No.: 18/534,628

(22) Filed: Dec. 9, 2023

(30) Foreign Application Priority Data
Sep. 8, 2023 (TW) 112134393

(51) Int. Cl. C05F 1/00; C05C 11/00; C05G 5/23

(52) U.S. Cl. CPC C05F 1/005; C05C 11/00; C05G 5/23

(57) ABSTRACT

Present invention teaches the method of using a keratin hydrolysis peptide (“KHP”) solution to increase the grape plant's production yield. By selectively choosing specific weights of feathers and water, and treating the mixture, though one embodiment does not have water mixed in, to a high-temperature high-pressure hydrolysis process, the resulting solution is confirmed to contain at least 253 peptides and then applied to the leaf surface of grape plants at different growth stages. Optionally, the KHP solution can be diluted by water, as taught in the specification, before applying to the leaf surface of the grape plants.

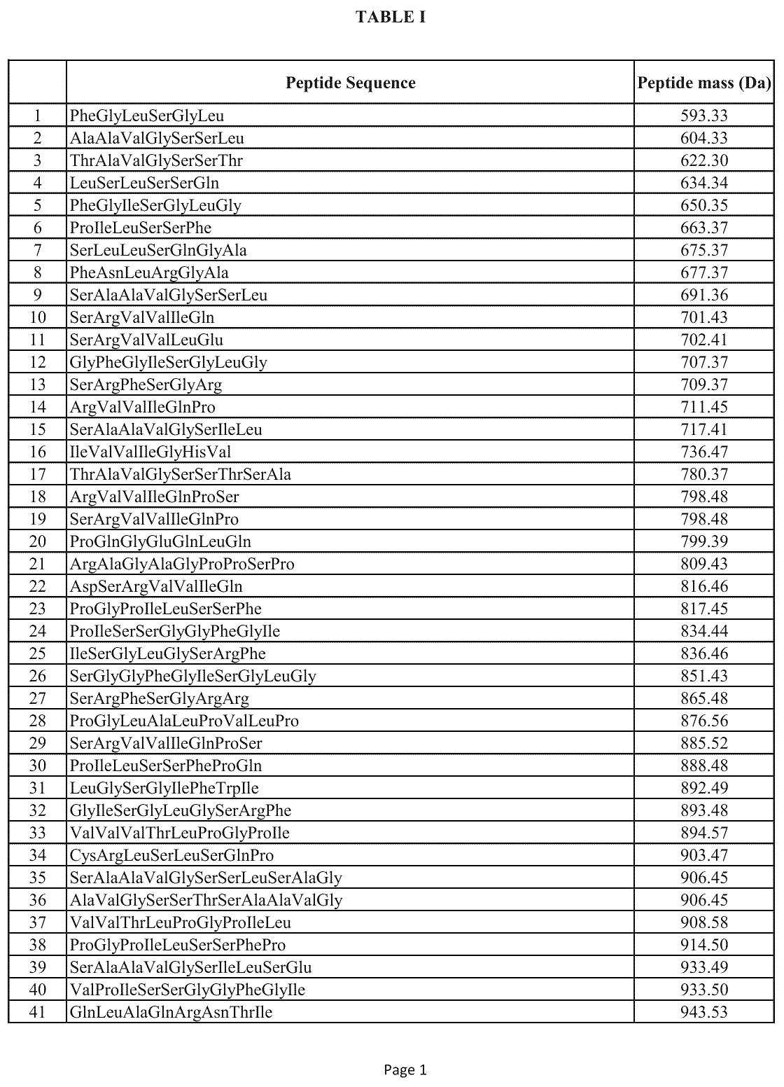

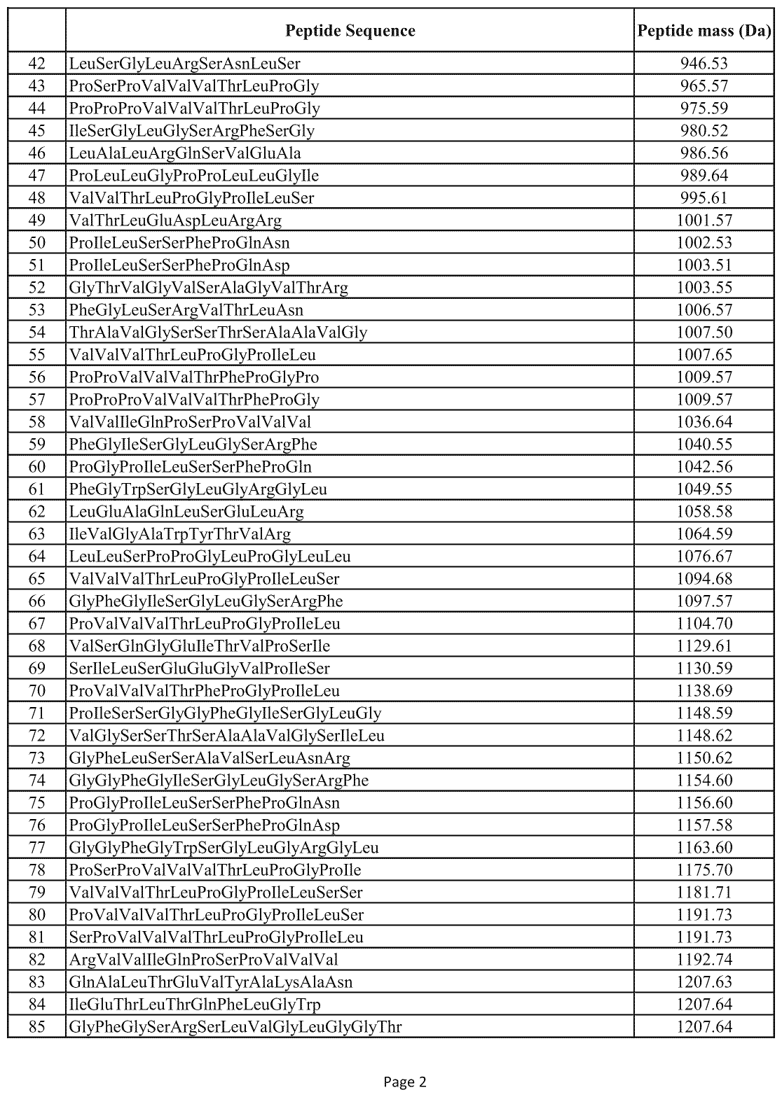

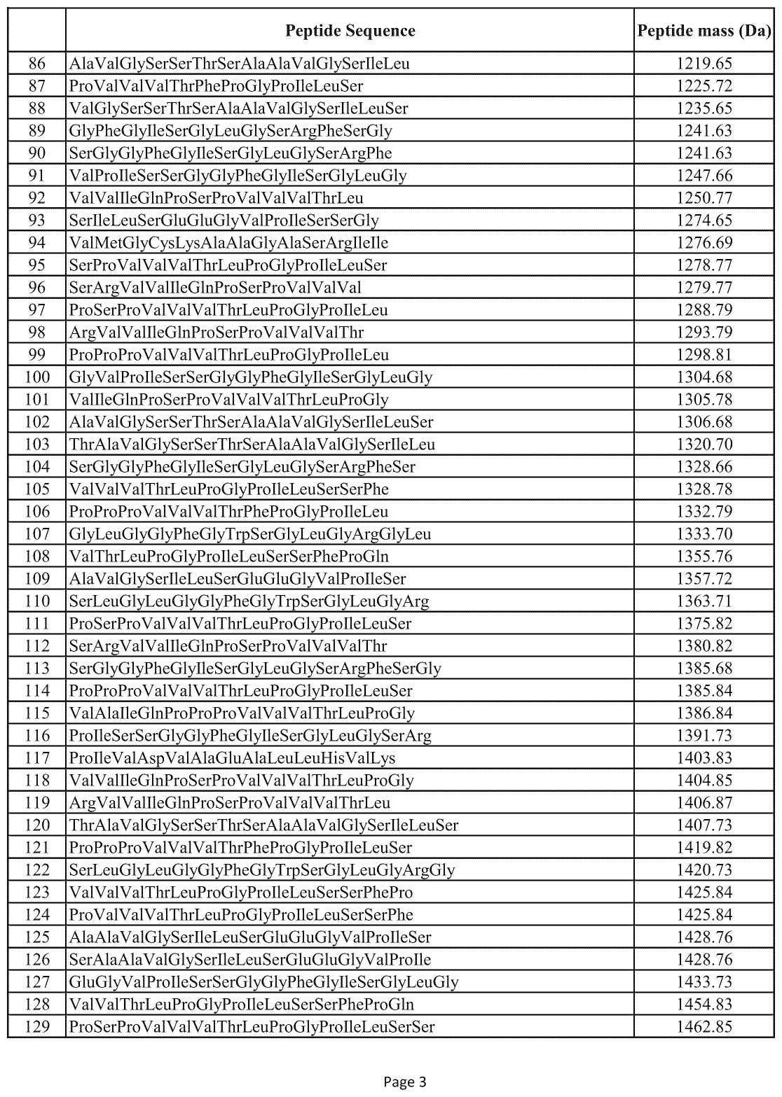

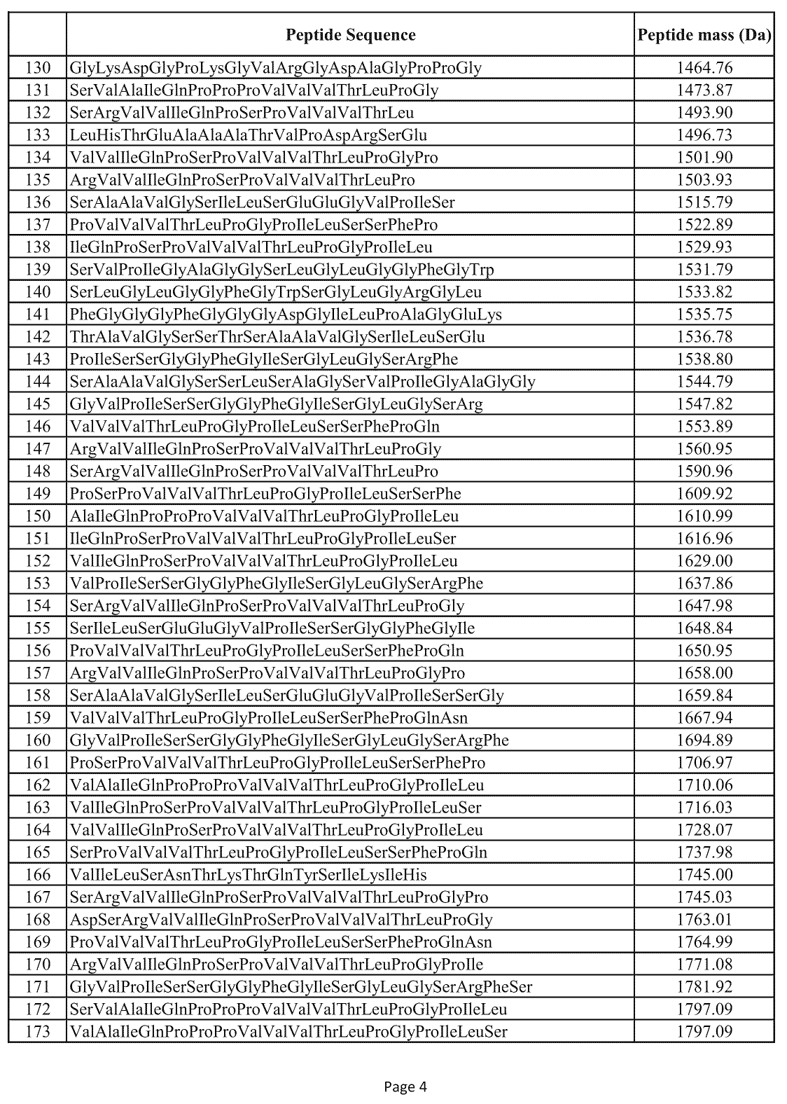

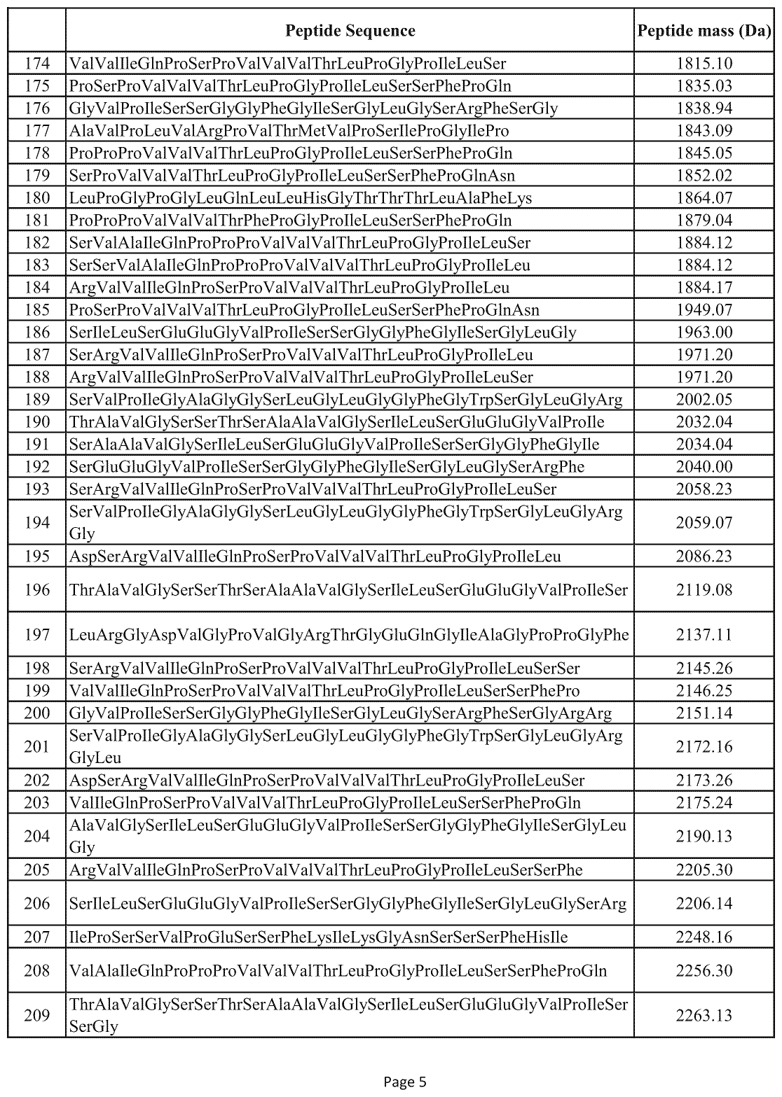

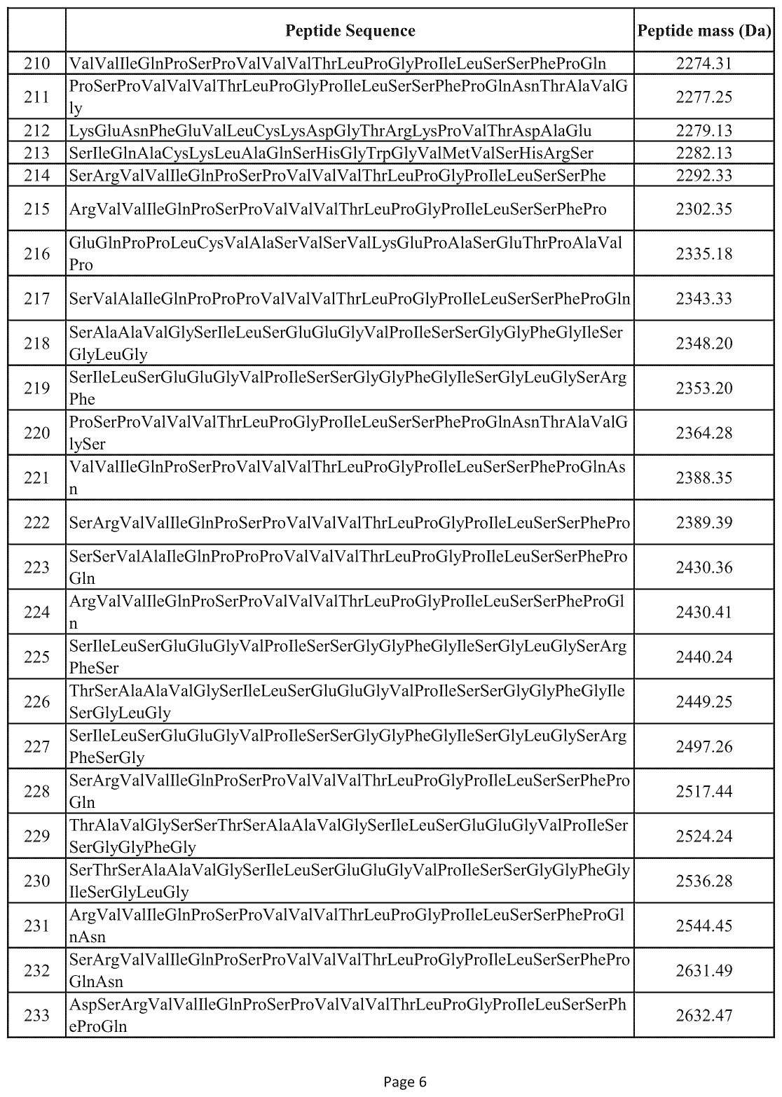

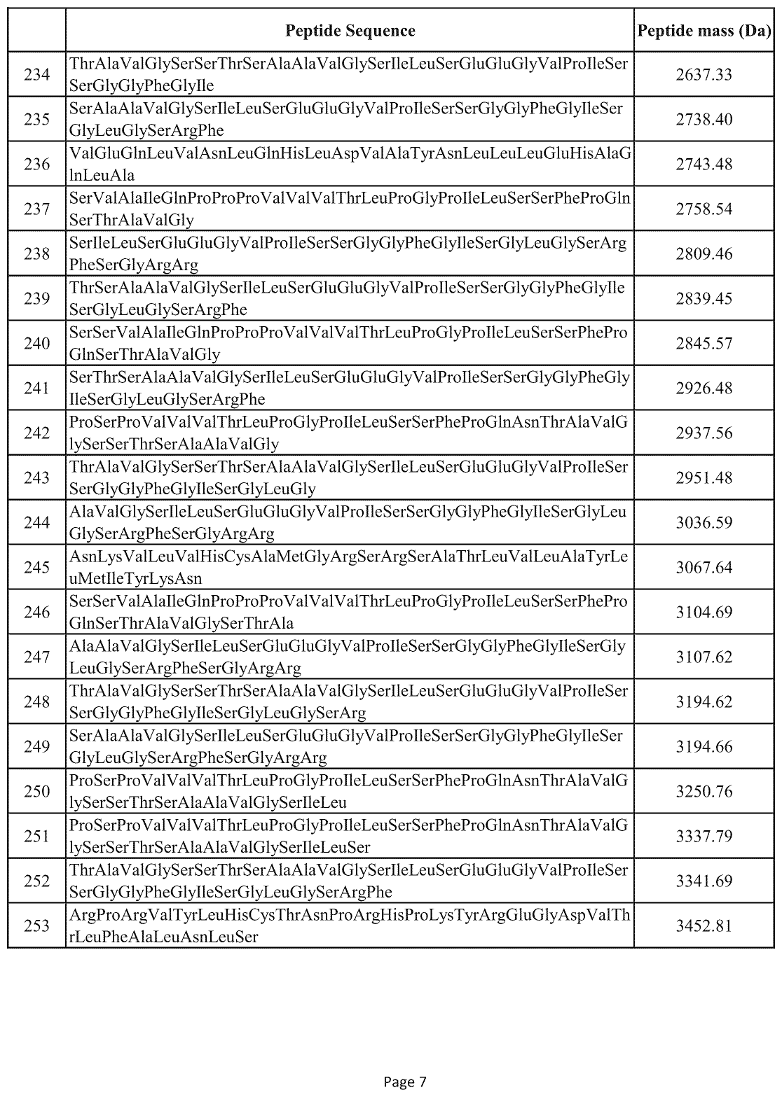

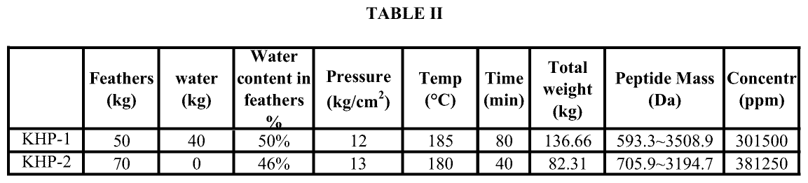

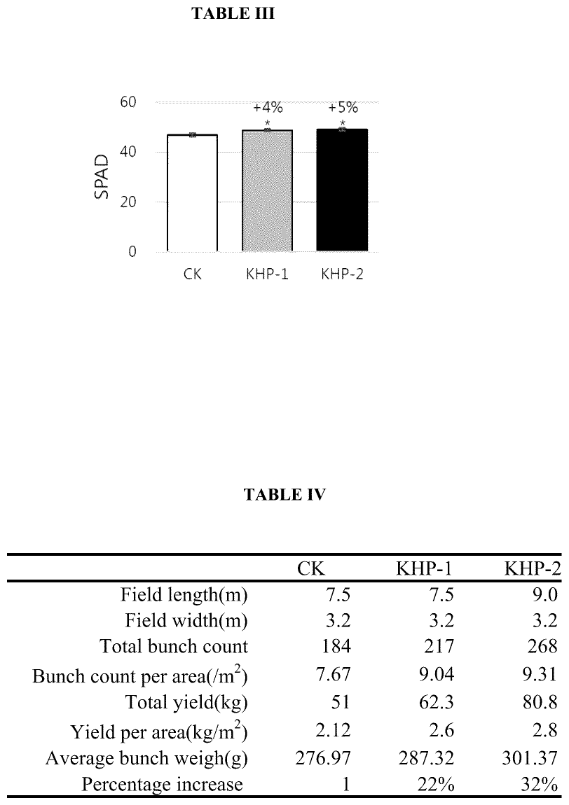

### PRIORITY CLAIM TO FOREIGN APPLICATION

Applicant hereby makes priority claim to a Taiwan application, number 112134393, having the Taiwan filing date of Sep. 8, 2023.

### BACKGROUND OF THE INVENTION

Present invention disclosed and claimed the method and application of a keratin hydrolysis peptide (“KHP”) solution to grape plants, increasing the production yield.

The KHP solution is made by a hydrolysis process using feathers and water, with one version of the solution using feathers only, via a high-temperature and high-pressure process, resulting in a solution that has many beneficial applications in the fields of horticulture, agriculture and potentially other farming businesses.

Hydrolyzed keratin has long been used to strengthen hairs, reduce breakage, and minimize damages. Such hydrolyzed keratin solution is also known to be applied to crops, in the hope of promoting the plants' health and increasing production yield by way of different method of applications. Some form of chemical process was attempted, and used, to break down the large protein molecule of the hydrolyzed keratin so that it can used in the intended direction/purpose. The chemical process, though, is generally not favored in the agricultural industry.

Grape is one of the largest fruit crops in the world. According to statistics, the global harvesting area reached 6,729,198 metric acers in 2021, with total production weight reaching 25 million metric tons.

Grape plants are generally divided into two categories: for direct consumption and for making wines. The grapes grown for direction consumption have been known to be more susceptible to weather changes, over exposure to sun (or under exposure), and, in recent years, the over application of fertilizers that, despite promoting the growth of leaves and foliage in general, lead to the reduction of actual yield.

The Taiwan grape plants' growth and production sometimes has an addition problem: incessant rains that deprived the plants of sufficient sun light for photosynthesis, which further lead to health issues and diseases inflicted upon grape plants.

Instead of relying on fertilizers, or KHP solutions done via chemical processes, to counter the problems such as those stated herein, the inventors of present application resort to the method of using a keratin hydrolysis peptide solution (KHP) to improve the grape's health and production yield.

It is well known that feathers of domestic birds/fowls contain 85-91% keratin, 13-15% organic nitrogen, 1.6-2% organic sulfur, as well as other materials. The high keratin content has drawn many prior researches that work to break down, by enzyme, chemical agents, or fermentation process, into peptides, amino acids and other smaller molecules that can be used for animal feeds, plant fertilizers, and cultivation bases.

Around 2019, Nurdiawati, et al, came up with a hydrolysis process, by mixing feathers and water (generally at a rate of 1:3) for treatment of a high-temperature and high-pressure (setting of 160° C., 6.1 kg/cm², or setting of 180° C. and 9.2 kg/cm² for a duration of 30 minutes) that obtained a solution. The keratin solution is then mixed with other fertilizers containing nitrogen, potassium and other minerals before applying to plants.

The keratin hydrolysis solution is shown to boost the growth of Pogostemon cablin and Vigna radiata, as reported by Nurdiawati et al., 2019.

Under the aegis of CH Biotech, inventors of present application experimented and worked on the method of employing a high-temperature and high-pressure hydrolysis process, materially different from Nurdiawati, to break down the keratin in feathers, and to form a hydrolyzed peptide (“KHP”) solution that can be applied to grape plants, suitable for increasing the fruiting weight and yield, increasing the more cost-effective grape farming and harvesting.

### SUMMARY OF THE INVENTION

The invention has two embodiments to create the KHP solution, where one version contains the water: feather weight ratio of 4:5, before mixing and placed into a sealed contained to go through the hydrolysis process. The second version does not add water, but simply uses feathers.

The first embodiment of the KHP solution is made by the steps of:
a. Preparing the KHP solution by mixing 50 kg of feathers whose content is 50% water and 40 kg of water in a sealed container;
b. hydrolyzing the mixture in the container with a temperature and pressure setting of 185° C. and 12 kg/cm² for a duration of 80 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to leaf surface of the grape plant at the fruit expansion stage.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

The method of using a keratin hydrolysis peptide (KHP) solution stated above where the solution is applied at the growth stage of fruit thinning.

The method of using a KHP solution is further diluted with water by volume at the ratio of 1:250-1,000 and is then sprayed to the grape plant leaves' surface at the growth stages as taught herein.

A second embodiment of the method of using a keratin hydrolysis peptide (KHP) solution comprising the steps of:
a. Preparing the KHP solution by preparing and putting 70 kg of feathers whose content is 46% water in a sealed container;
b. hydrolyzing the mixture in the container with a temperature and pressure setting of 180° C. and 13 kg/cm² for a duration of 40 minutes;
c. using a mass spectrometer to confirm the combination of peptides in the solution to contain at least 253 peptides as listed in the specification where their molecular masses are between 500 and 4,000 Daltons, and the concentration is in the range of 2.0×10⁵˜4.5×10⁵ ppm; and
d. applying the solution to the leaf surface of the grape plant at the fruit expansion stage.

The confirmation of some of the bioactive peptides is further done by referencing the BIOPEP-UWM database.

The method of using a keratin hydrolysis peptide (KHP) solution stated can be applied to the leaf surface of the grape plant at the fruit thinning stage.

Similar to the first embodiment, the method of using a KHP solution can be further diluted with water by volume at the ratio of 1:250-1,000 or more specifically at the ratio of 1:300-500, at the stages as taught herein.

To prove the efficacy of the method of increasing grape production yield, the inventors conducted experiments in the field and presented the results herein.

Three groups of grape plants were tested, noted as CK group, KHP-1 group and KHP-2 group. The CK group is the check group where no KHP solution is applied; the normal amount of water was given to the CK group, in accordance with the normal growth cycles of the grape plant in question. The other two groups are applied with the first and second embodiment KHP solutions noted as KHP-1 and KHP-2.

The KHP solutions are sprayed to the leaves' surface at the fruit expansion stage and then at fruit thinning stage.

A chlorophyll SPAD reading is taken to compare the growth status of the grape plants in the three groups. The result is presented in Table III.

At harvest time, six (6) bunches of better-looking grape fruits from each group and then their production parameters are compared and noted in Table IV.

As shown in Tables III and IV, the beneficial use of the method of KHP solutions, both embodiments, increased the production yield of the grape plants substantially, as well be further discussed in later sections.

### BRIEF DESCRIPTION OF DRAWINGS

The accompanying drawings, figures and tables, which are incorporated in and constitute a part of this specification, illustrate and exemplify the preferred embodiments of the invention. Together with the description, serve to explain the principles of the invention.

Table I shows the at least 253 peptides and its annotated sequences for the solution generated in accordance with the disclosure of this application.

Table II shows parameters for the two KHP solution embodiments.

Table III shows the chlorophyll count measured by SPAD readings among the three (3) groups of grape plants a s tested in the field.

Table IV shows the comparison of the grape plant yield as tested in the field.

### DETAILED DESCRIPTION OF THE INVENTION

The method of using a keratin hydrolysis peptide (KHP) solution to improve grape plant's production yield, comprising the steps of:
a. mixing 40 kg of water and 50 kg of feathers together, wherein the feathers are of 50% water content;
b. stirring the feathers and water mixture in a sealed container;
c. hydrolyzing the mixture in the container with a temperature and pressure setting of 185° C. and 12 kg/cm² for a duration of 80 minutes;
d. using a mass spectrometer to confirm the combination of peptides in the solution to contain at least 253 peptides as listed in the specification whereby their molecular masses are between 500 and 4,000 Dalton, and consisting of between 5 and 30 amino acids.
e. the solution is further filtered and concentrated to the range of 300,000-450,000 ppm concentration.
f. Applying water to dilute the solution to grape leaves' surface during a grape plant's fruit expansion period.

To confirm the 253 peptides, the inventors used a Dionex UltiMate 3000 UPLC to separate the peptides; an analysis is done via Thermo Orbitrap Fushion Lumos Tribrid Orbitrap mass spectrometry to identify the peptides, which are then subsequently confirmed by looking up the BIOPEP-UWM database.

A second embodiment of the KHP solution is done by using feathers only, without water, by the steps of:
a. putting 70 kg of feathers in a sealed container, wherein the feathers are of 46% water content;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 500 and 4,000 Dalton, and consisting of between 5 and 30 amino acids.
d. the solution is further filtered and concentrated to the range of 300,000-450,000 ppm concentration.
e. Applying water to dilute the solution to grape leaves' surface during a grape plant's fruit expansion stage.

The confirmation of some of the bioactive peptides is further done by referencing the BIOPEP-UWM database.

The method of using keratin hydrolysis peptide (KHP) solutions above, both embodiments, where the solution(s) can be applied to the leaves' surface at fruit thinning stage.

The method of using KHP solutions above where the solutions, both embodiments, can be diluted with water by volume at the ratio of 1:250-1,000 and sprayed to the leaves' surface at the growth stages fruit expansion and/or fruit thinning.

The method of using keratin hydrolysis peptide (KHP) solutions above where the solutions, both embodiments, can be more specifically diluted with water by volume at the ratio of 1:300-500 and sprayed to the leaves' surface at the growth stages of fruit expansion and/or fruit thinning.

To prove the efficacy of the method of increasing grape production yield, the inventors conducted experiments in the field and presented the results herein. The species of Vitis Vinifera Lin, Kyoho is selected for the field tests done in Changhua County, Taiwan.

The selected grape plants are for consumers' direct consumption and are around 20 years old, generally the mature and best age group for conducting scientific tests such as the ones herein.

Three groups of grape plants were tested, noted as CK group, KHP-1 group and KHP-2 group. The CK group is the check group where no KHP solution is applied. The other two groups are applied with the first and second embodiment KHP solutions, diluted at 1:500 ratio. The tests went on for about six (6) months. first embodiment solution is noted as KHP-1 and the second embodiment is noted as KHP-2.

The KHP solutions are sprayed to the leaves' surface at the fruit expansion stage and then at fruit thinning stage.

Roughly seven (7) days after the application of KHP solution at the fruit expansion stage, a chlorophyll SPAD reading is taken to compare the growth status of the grape plants in the three groups. As can be shown in Table III, both KHP-1 and KHP-2 groups show substantial increase in the plants' photosynthesis capacity due to higher chlorophyll counts than the check (CK) group.

At harvest time, six (6) bunches of better-looking grape fruits from each group and then their production parameters are compared and noted in Table III.

As shown in Table III, the production yield increases per unit planting are increased by 22% in the KHP-1 group and 32% in the KHP-2 group. The average per-bunch weights are 287.32 k (KHP-1) and 301.37 g (KHP-2), compared to the CK group.

Other performance figures also show the beneficial result of the method of applying the KHP solutions as taught herein.

Overall, the research and experiment show that the method of making the KHP solutions and the applications disclosed herein substantially add to the grape plants' health and production yield as intended by the inventors of the present application.

While the disclosure herein gave limited teachings and embodiment examples, it should be noted that the description and disclosure made herein illustrated the preferred embodiments of the invention and are not meant to limit the scope of the applicant's rights. Variations and alterations may be employed for yet additional embodiments without departing from the scope of the invention herein.

1. The method of using a keratin hydrolysis peptide (KHP) solution to improve grape plant's production yield, comprising the steps of:
a. mixing 40 kg of water and 50 kg of feathers together, wherein the feathers are of 50% water content;
b. stirring the feathers and water mixture in a sealed container;
c. hydrolyzing the mixture in the container with a temperature and pressure setting of 185° C. and 12 kg/cm² for a duration of 80 minutes;
d. using a mass spectrometer to confirm the combination of peptides in the solution to contain at least 253 peptides as listed in the specification whereby their molecular masses are between 500 and 4,000 Dalton, and consisting of between 5 and 30 amino acids;
e. the solution is further filtered and concentrated to the range of 300,000-450,000 ppm concentration; and
f. Applying water to dilute the solution to grape leaves' surface during a grape plant's fruit expansion stage.

2. The method of claim 1 wherein the solution is applied to grape leaves' surface during the grape plant's fruit thinning stage.

3. The method of claim 1 wherein the amount of water used to dilute the solution has a volume ratio of 125-1,000 (water):1 (solution).

4. The method of claim 1 wherein the amount of water used to dilute the solution has a volume ratio of 300-500 (water):1 (solution).

5. The method of using a keratin hydrolysis peptide (KHP) solution to improve grape plant's production yield, comprising the steps of:
a. putting 70 kg of feathers in a sealed container, wherein the feathers are of 46% water content;
b. hydrolyzing the feathers in the container with a temperature and pressure setting of 180° C. and 13 kg/cm² for a duration of 40 minutes;
c. using a mass spectrometer to confirm the combination of peptides in the solution to contain at least 253 peptides as listed in the specification whereby their molecular masses are between 500 and 4,000 Dalton, and consisting of between 5 and 30 amino acids;
d. the solution is further filtered and concentrated to the range of 300,000-450,000 ppm concentration; and
e. Applying water to dilute the solution to grape leaves' surface during a grape plant's fruit expansion stage.

6. The method of claim 5 wherein the solution is applied to grape leaves' surface during a grape plant's fruit thinning stage.

7. The method of claim 5 wherein the amount of water used to dilute the solution has a volume ratio of 125-1,000 (water):1 (solution).

8. The method of claim 5 wherein the amount of water used to dilute the solution has a volume ratio of 300-500 (water):1 (solution).
